(19) United States
(12) Patent Application Publication
(10) Pub. No.: US 2018/0104396 A1
(43) Pub. Date: Apr. 19, 2018

(54) BREAST PUMP

(71) Applicant: Cho Hee PARK, Anseong-si, Gyeonggi-do (KR)

(72) Inventor: Cho Hee PARK, Anseong-si, Gyeonggi-do (KR)

(21) Appl. No.: 15/567,392

(22) PCT Filed: May 18, 2016
(86) PCT No.: PCT/KR2016/005271

§ 371 Date: Oct. 18, 2017

(30) Foreign Application Priority Data
May 19, 2015 (KR) 10-2015-0069851

(51) Int. Cl. A61M 1/06

(52) U.S. Cl. CPC A61M 1/062; A61M 39/10; A61M 1/066

(57) ABSTRACT

A breast pump includes: a close-contact housing brought into contact with a breast of a lactating woman and having a protruding part, and a milk discharge hole formed on an end-side bottom surface of the protruding part; a cap connector covering an end side of the protruding part, having a spacing space between the cap connector and the protruding part, and provided with a milk discharge tube; a funnel connector on an upper side of the cap connector to have an inner space, and provided with a communication flow passage through which the inner space and the spacing space communicate; a cover to cover the inner space of the funnel connector; a flow separation membrane for vertically separating a space between the funnel connector and the cover; and an air tube through which air is allowed to flow into/out of the space between the cover and the flow separation membrane.

### TECHNICAL FIELD

The present invention relates to a breast pump, and more particularly, to a breast pump configured to enable a lactating woman to freely use both hands during milking and to prevent breast milk from flowing backward.

[symbol]

### BACKGROUND ART

In general, breast pumps (so called, milk extractors) are used to forcibly extract excessively remaining milk from breasts because lactating women feel strong pain (breast congestion) due to the excessively remaining milk when breast milk remains in breasts which feed the breast milk to infants, or the infants do not take the breast milk.

Such breast pumps are sold in various types on the market at present and are roughly classified into a manual type and an electrical type according to power sources. The manual breast pumps each include a funnel-shaped suctioner worn on a breast, a rubber-ball-shaped vacuum pump for generating an air pressure in the suctioner; and a milk bottle for storing breast milk introduced into the suctioner. In the manual breast pumps including the above-mentioned configuration, a lactating woman or an assistant repeats an operation of bringing the suctioner into close contact with a breast by one hand and pressing and releasing the vacuum pump by the other hand, thereby forcibly extracting breast milk from the breast. However, such manual breast pumps have a very low milk extracting efficiency because a strong air pressure can not be generated due to the air pressure generated by the hands of a user, and have substantial inconvenience in that the user consumes a great amount of power.

On the other hand, electric breast pumps include an air pressure generating pump having a motor and an air cylinder, a funnel-shaped suctioner connected to the air pressure generating pump and worn on a breast of a lactating woman, and a milk bottle storing breast milk introduced through the suctioner, thereby forcibly extracting the breast milk by using the suctioning force generated by the air pressure generating pump. When such electric breast pumps are used, there are merits in that a user does not consume a great amount of force and the milk extracting efficiency is improved.

However, when typical breast pumps are used, a lactating woman should continuously hold a milk bottle, and thus, there is a limitation in that much inconvenience is involved in extracting milk. In addition, a limitation frequently occurs in that a breast milk back flow is caused while the breast milk is suctioned through the suctioner, a pipe passage is blocked due to the back flow, and operational troubles are caused due to the contamination of a pump. That is, when the breast milk is suctioned by using the breast pumps, the breast milk is extracted from breasts while the pressure inside a suctioner is rapidly decreased by the operation of a pump. In this case, since a strong negative pressure is applied also to a pipe passage connecting the suctioner and the pump, a portion of the breast milk suctioned into the suctioner occasionally flows backward to the pipe passage.

[symbol]

### DISCLOSURE OF THE INVENTION

### Technical Problem

In order to address the above-mentioned limitations, the present invention provides a breast pump which enables a lactating woman to freely use both hands during milking, is capable of preventing breast milk from flowing backward, and is easily cleaned and maintained.

[symbol]

### Technical Solution

A breast pump for achieving the above-mentioned purpose according to the present invention includes: a close-contact housing brought into close contact with a breast of a lactating woman and having a protruding part which extends forward and is formed in a portion corresponding to a nipple of the lactating woman and a milk discharge hole formed on an end-side bottom surface of the protruding part; a cap connector coupled in a structure covering an end side of the protruding part, having a spacing space between the cap connector and an end surface of the protruding part, and provided with a milk discharge tube which extends downward from a lower end of the milk discharge hole and communicates with the spacing space; a funnel connector provided on an upper side of the cap connector so as to have a concavely formed upper surface and to thereby have an inner space, and provided with a communication flow passage through which the inner space and the spacing space communicate with each other; a cover coupled so as to cover the inner space of the funnel connector; a flow separation membrane for vertically separating a space between the funnel connector and the cover; and an air tube through which air is allowed to flow into/out of the space between the cover and the flow separation membrane.

[symbol]

In an embodiment, the breast pump may further include a storing housing coupled to the close-contact housing in a detachable structure so as to house therein the cap connector, the funnel connector, the cover, the flow separation membrane, and a silicon valve, and provided with a space in which breast milk discharged through the breast milk discharge tube is stored.

[symbol]

In an embodiment, a cleaning hole communicating with the communication flow passage may be formed on an outer end of the cap connector, and

the storing housing may be provided with a sealing packing pressed to the cleaning hole when coupled to the close-contact housing.

[symbol]

In an embodiment, the cap connector may be provided with a milk bottle connector on a side in which the breast milk discharge tube is formed.

[symbol]

In an embodiment, the breast pump may further include a silicon valve having one side coupled to an outlet side of the breast milk discharge tube and the other side which is sharply formed and provided with a cut line.

[symbol]

In an embodiment, the breast pump may further include an electric pump for suctioning air through the air tube.

[symbol]

### Advantageous Effects

The breast pump according to the present invention has merits of having excellent usability because a lactating woman can freely use both hands, having sanitariness because the phenomenon of breast milk back flow can be prevented, and having convenience in use due to easy cleaning and maintenance.

[symbol]

### BRIEF DESCRIPTION OF THE DRAWINGS

FIG. 1 is a perspective view of a breast pump according to the present invention.

FIGS. 2 to 4 are exploded perspective views in each direction of a breast pump according to the present invention.

FIG. 5 is an exploded cross-sectional view of a breast pump according to the present invention.

FIG. 6 is a front view of a breast pump in use according to the present invention.

FIGS. 7 and 8 are cross-sectional views of a breast pump in use according to the present invention.

FIG. 9 is an exploded cross-sectional view of a breast pump according to a second embodiment of the present invention.

FIG. 10 is an exploded perspective view of a breast pump according to a second embodiment of the present invention.

FIG. 11 is a perspective view of a breast pump according to a second embodiment of the present invention.

[symbol]

### MODE FOR CARRYING OUT THE INVENTION

Hereinafter, embodiments of a breast pump according to the present invention will be described in detail with reference to the accompanying drawings.

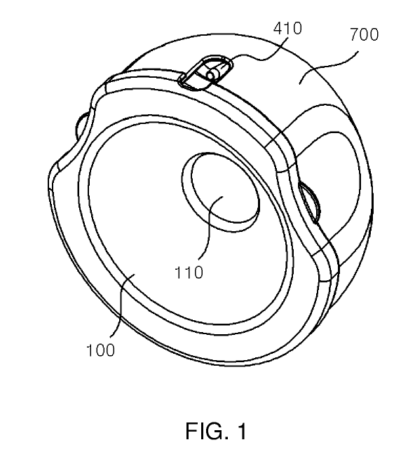

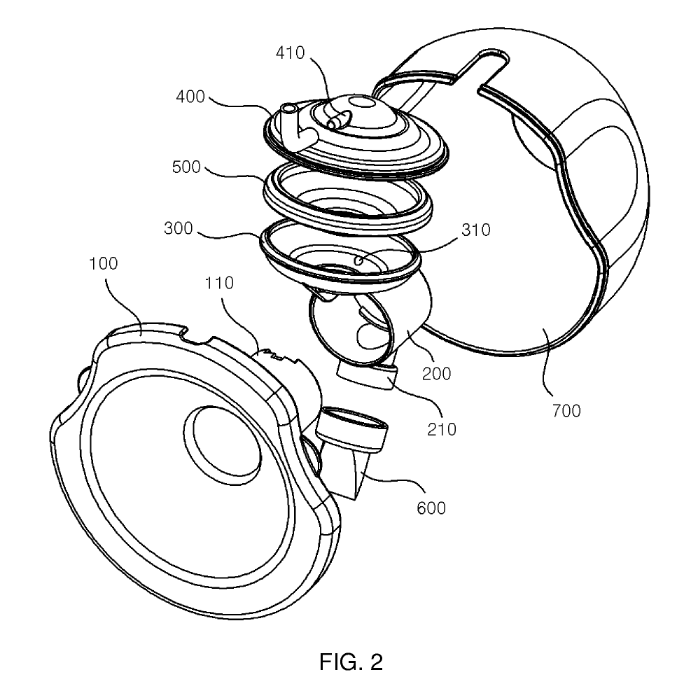

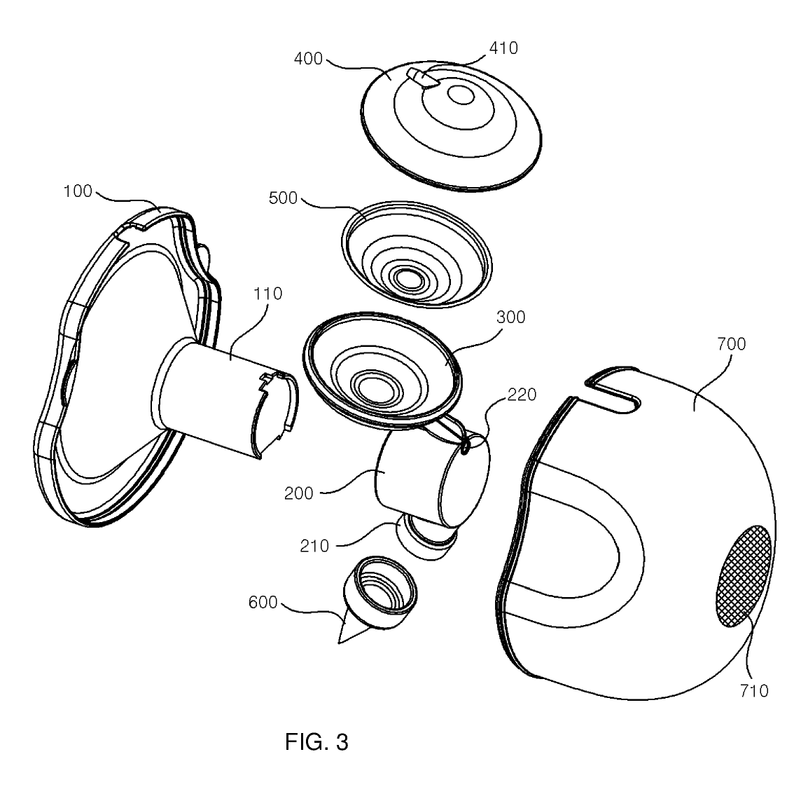

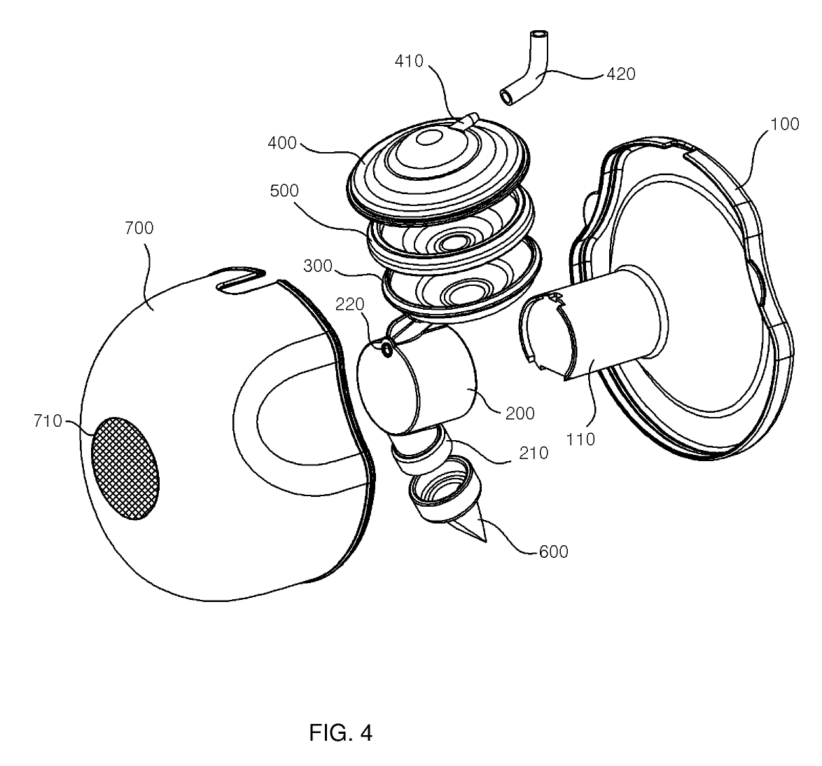

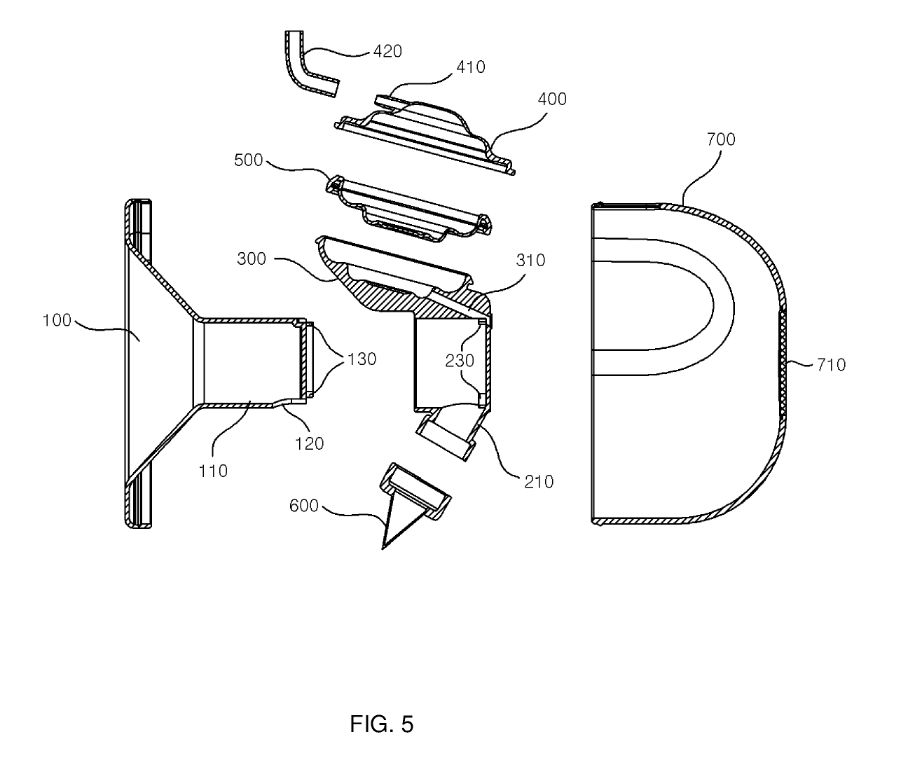

FIG. 1 is a perspective view of a breast pump according to the present invention, FIGS. 2 to 4 are exploded perspective views in each direction of a breast pump according to the present invention, and FIG. 5 is an exploded cross-sectional view of a breast pump according to the present invention.

A breast pump according the present invention is an apparatus for extracting breast milk by applying a vacuum pressure to the side of breasts 10 of a lactating woman and is characterized by being configured to be capable of preventing a phenomenon in which the breast milk flows backward through a flow passage to which the vacuum pressure is applied.

That is, the breast pump according to the present invention includes: a close-contact housing 100 having a protruding part 110, which extends forward and is formed in the portion corresponding to a nipple of a lactating woman, and a breast milk discharging hole 120 formed on an end-side bottom surface of the protruding part 110; a cap connector 200 coupled in a structure covering an end side of the protruding part 110; a funnel connector 300 having an upper surface concavely formed so as to have an inner space and thereby connected to an upper side of the cap connector 200; a cover 400 coupled so as to cover the inner space of the funnel connector 300; a flow separation membrane 500 for vertically separating the space between the funnel connector 300 and the cover 400; an air tube 420 for allowing air to flow into/out of the space between the cover 400 and the flow separation membrane 500; and an electric pump 800 for suctioning air through the air tube 420.

The cap connector 200 has a portion corresponding to an end surface of the protruding part 110 which is not brought into close contact with the end of the protruding part 110 but spaced apart a predetermined distance from the end of the protruding part 110, and thus, provided with a spacing space G between the end surface of the protruding part 110 and the cap connector 200 and a breast milk discharge tube 210 extending downward from a lower end of the breast milk discharge hole 120 and communicating with the spacing space G. In addition, a communication flow passage 310 through which the inner space of the funnel connector 300 and the spacing space G communicates with each other is provided, wherein the inner space of the funnel connector 300 communicates with the inner side of the protruding part 110 through the communication flow passage 310, the spacing space G, and the breast milk discharge tube 210.

The flow separation membrane 500 is formed of a ductile material so as to be easily bendable by external force. Thus, when air between the cover 400 and the flow separation membrane 500 is suctioned to the outside through an air nozzle 410, the flow separation membrane 500 is moved upward. As such, when the flow separation membrane 500 is moved upward, the space between the flow separation membrane 500 and the funnel connector 300 is expanded to form a vacuum pressure. The vacuum pressure is transferred to a breast 10 of a lactating woman through the communication flow passage 310, the spacing space G, the breast milk discharge hole 210, and the inside of the protruding part 110, and thus, breast milk is extracted from the breast 10 of the lactating woman.

Here, there may be a case in which the breast milk of the lactating woman is suctioned up to the inside of the funnel connector 300 through the spacing space G and the communication flow passage 310 by the vacuum pressure, but the inner space of the funnel connector 300 is sealed by the flow separation membrane 500, and thus, the breast milk is not allowed to flow backward up to the air nozzle 410. That is, the breast pump according to the present invention does not allow breast milk to flow backward to thereby be discharged to the outside, and thus, has a merit in that there is no phenomenon in which the electric pump 800 is contaminated or damaged by the breast milk. In addition, since breast milk is not discharged to the outside, it is possible to achieve an effect of improving sanitary conditions.

[symbol]

Meanwhile, the breast pump according to the present invention may be provided with a storing housing 700 coupled to the close-contact housing 100 in a structure in which the cap connector 200, the funnel connector 300, the cover 400, the flow separation membrane 500, and a silicon valve 600 are housed in the storing housing 700 so as to collect extracted breast milk even without using a separate milk bottle. As such when the storing housing 700 is attached to the close-contact housing 100, there is a merit in that a lactating woman may use both hands because the lactating woman can extract breast milk even without holding a separate milk bottle. Of course, the storing housing 700 is coupled to the close-contact housing 100 in a detachable structure, and thus, a user can collect breast milk by separating the storing housing 700 from the close-contact housing 100 after a certain amount of breast milk is stored in the storing housing 700.

Of course, even without separating the storing housing 700 from the close-contact housing 100, a separate breast milk discharge opening may be provided in the storing housing 700 so as to be capable of collecting the breast milk by discharging the breast milk in the storing housing 700 to the outside. Here, a lid which can be opened or closed depending on the selection of the user should be provided in the breast milk discharge opening. Such a lid has been widely used in various forms in various technical fields relating to the present invention, and thus, detailed description on the structure and operations of the lid will not be provided.

In addition, a silicon valve 600 having a check valve function is favorably attached to the outlet side of the breast milk discharge tube 210 so as to prevent a phenomenon in which the breast milk filled in the storing housing 700 flows backward through the breast milk discharge tube 210. The silicon valve 600 has one side (in the current embodiment, an upper side) coupled to the breast milk discharge tube 210 and the other side (in the current embodiment, a lower side) formed in a sharp shape such that a cut line (Not shown) is formed on the other side. Accordingly, the breast milk discharged through the breast mile discharge tube 210 is introduced in the silicon valve 600, and is then filled in the storing housing 700 through the cut line. However, since it is difficult to introduce the breast milk in the storing housing 700 into the silicon valve 600 through the cut line, it is possible to prevent the phenomenon in which the breast milk in the storing housing 700 flow backward to the breast 10 of the lactating woman.

Meanwhile, as described above, the breast milk in the protruding part 110 may be introduced up to the communication flow passage 310 by a vacuum pressure. When the communication flow passage 310 is not exposed to the outside of the cap connector 200, it is difficult to clean the communication flow passage 310 and thus, there is a concern about contamination and propagation of bacteria. Accordingly, a cleaning hole 220 communicating with the communication flow passage 310 may be formed on an outer end of the cap connector 200.

Here, when the cleaning hole 220 is always opened, there is concern that the vacuum pressure formed in the communication flow passage 310 is released through the cleaning hole 220, and thus, the storing housing 700 is favorably provided with a sealing packing 710 for sealing the cleaning hole 220 in such a manner that the storing housing 700 is pressed to the cleaning hole 220 when coupled to the close-contact housing 100. The sealing packing 710 should be formed of a material having a sealing capability such as rubber. Meanwhile, since the remaining portion of the storing housing 700 except for the sealing packing 710 is favorably formed of plastic so as to have a strength of at least a reference value, the storing housing 700 is favorably formed through a double extraction process.

In addition, in order that the coupling direction of the protruding part 110 and the cap connector 200 may be constantly limited, mounting grooves 130 are formed on an end of the protruding part 110, and mounting protrusions 230 fitted into the mounting grooves 130 are formed on an inner side of the cap connector 200. As such, when the mounting grooves 130 and the mounting protrusions 230 are formed, the phenomenon in which the cap connector 200 is freely rotated with respect to the protruding part 110 does not occur, and thus, there is a merit in that it is possible to more stably extract breast milk.

In addition, the close-contact housing 100 and the storing housing 700 favorably have non-circular horizontal cross-sections so that the coupling angle therebetween may be maintained and the close-contact housing 100 and the storing housing 700 may be more firmly grasped and rotated by a user. However, the shapes of the close-contact housing 100 and the storing housing 700 may be changed into various shapes aside from the shapes illustrated in the current embodiment.

Meanwhile, in the current embodiment, although the structure in which air is suctioned through the air tube 420 by the electric pump 800, that is, an automatic breast pump in which a vacuum pressure is generated by the electric pump 800 is illustrated, a manual breast pump in which a tube pump is mounted so as to form a vacuum pressure by pressing the tube pump by a hand of a user instead of the electric pump 800 may be provided.

[symbol]

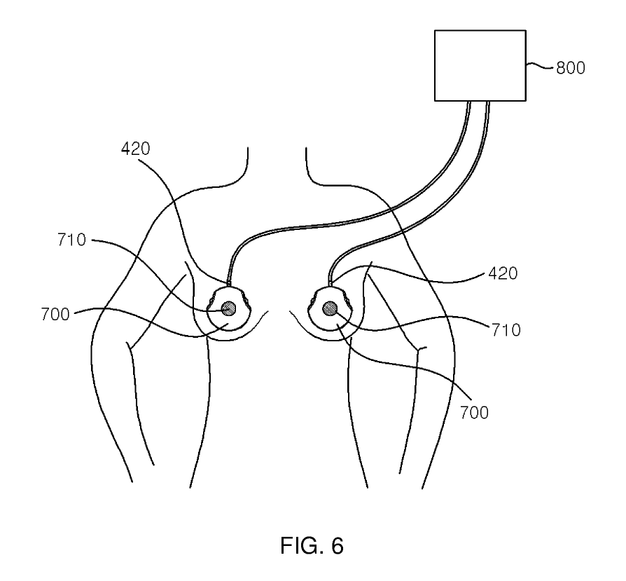

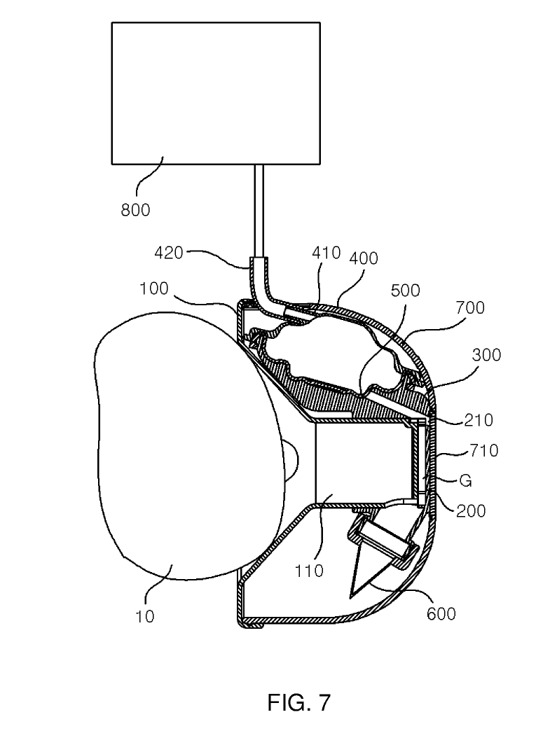

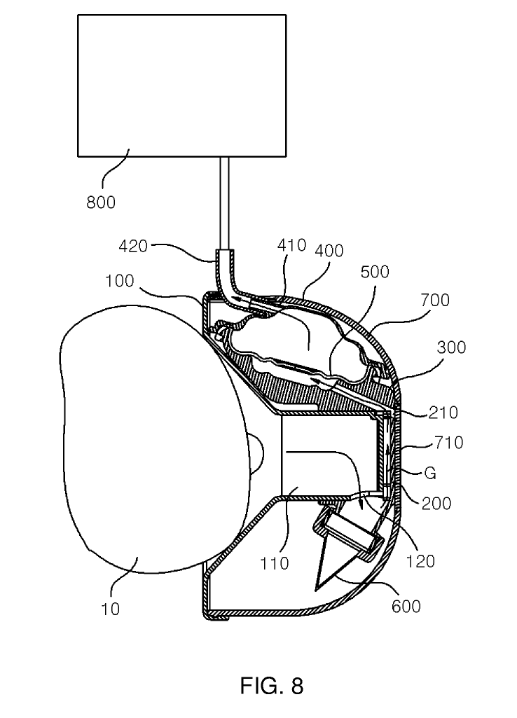

FIG. 6 is a front view of a breast pump in use according to the present invention, and FIGS. 7 and 8 are cross-sectional views of a breast pump in use according to the present invention.

Breast pumps according to the present invention are independently mounted on each of left and right breasts as illustrated in FIG. 6. When the breast pumps according to the present invention are used, it is possible to extract breast milk simultaneously from the left and right breasts, and thus there is merits in that the milk extraction time can be reduced by half and both hands of a lactating woman become free because it is unnecessary for the lactating woman to hold the breast pump by the hand during milk extraction.

Meanwhile, the breast pump according to the present invention maintains a state in which a flow separation membrane 500 is mounted on the bottom surface of a funnel connector as illustrated in FIG. 7, during an initial stage in which an electric pump 800 is not operated, that is, a vacuum pressure is not formed inside from a communication flow passage 310 to a protruding part 110.

In a state illustrated in FIG. 7, when the electric pump 800 is operated and then air between a cover 400 and the flow separation membrane 500 is discharged through an air tube 420 and an air nozzle 410, the flow separation membrane 500 is pulled toward the cover 400 as illustrated in FIG. 8, and then, a space is formed between the flow separation membrane 500 and the funnel connector 300, and thus, a vacuum pressure is generated in the communication flow passage 310. The vacuum pressure generated in the communication flow passage 310 is applied into the protruding part 110 through a spacing distance G and a breast discharge hole 120, a stimulus is transferred to a breast 10 closely contacting a close-contact housing 100, and thus, it is possible to extract milk.

Meanwhile, although breast milk being discharged through a breast milk discharge tube 210 may be suctioned up to the inside of the funnel connector 300 through the spacing space G and the communication flow passage 310 by the vacuum pressure, the funnel connector 300 and the cover 400 are completely separated by the flow separation membrane 500, and thus, the phenomenon in which the breast milk introduced into the funnel connector 300 is suctioned into the air tube 420 through the air nozzle 410 does not occur.

In addition, when the operation of the electric pump 800 is stopped and thereby, the vacuum pressure formed between the cover 400 and the flow separation membrane 500 is released, the flow separation membrane 500 is returned to an original state of being in close-contact with the bottom surface of the funnel connector 300 by a self elastic recovering force, that is, to the state illustrated in FIG. 8. As such, the flow separation membrane 500 which is deformed when an external force is applied and is returned to the original state when the applied force is released is used for various usages in hydraulic fields. Thus, detailed descriptions on the material and the recovering principle of the flow separation membrane 500 will not be provided.

[symbol]

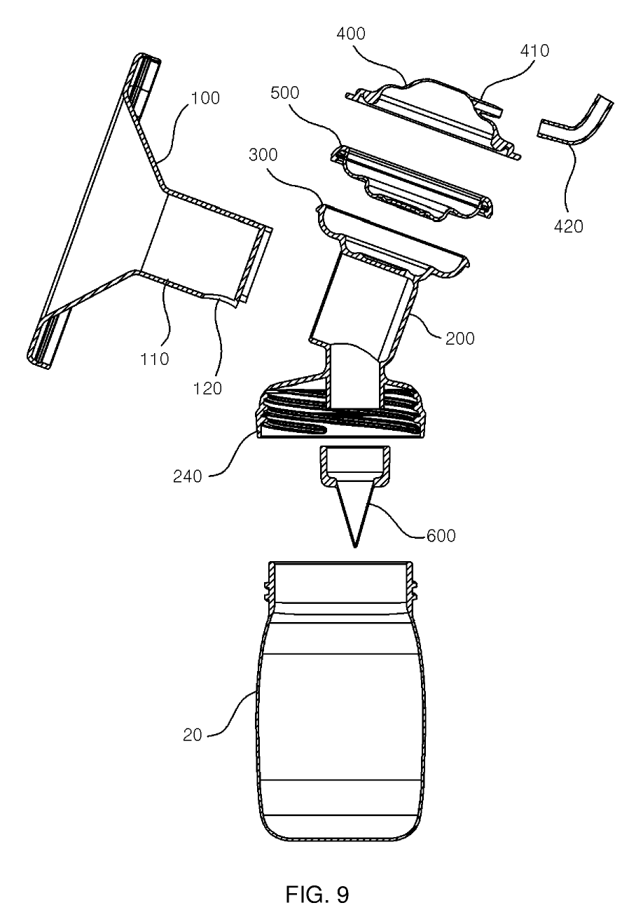

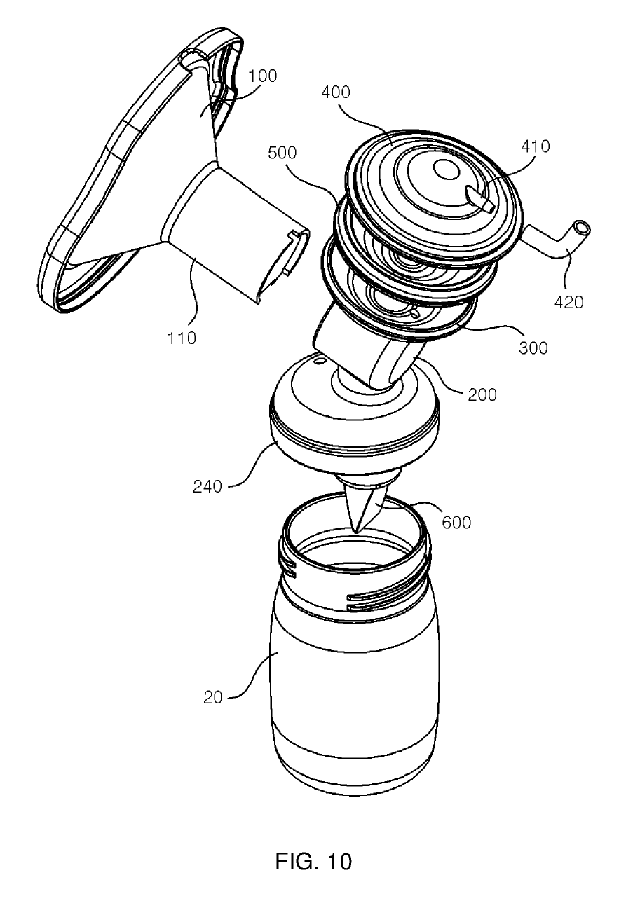

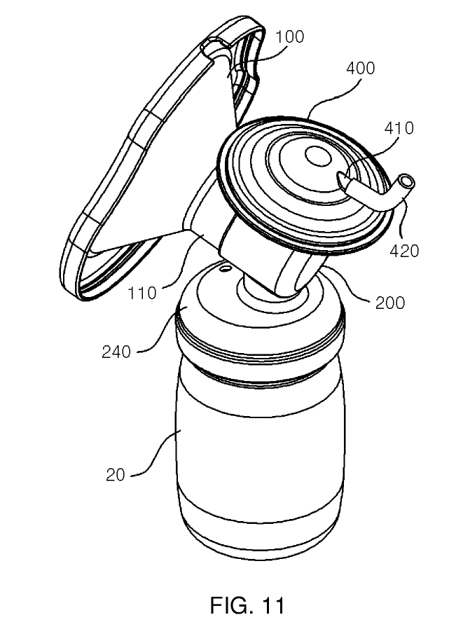

FIG. 9 is an exploded cross-sectional view of a breast pump according to a second embodiment of the present invention, FIG. 10 is an exploded perspective view of a breast pump according to a second embodiment of the present invention, and FIG. 11 is a perspective view of a breast pump according to a second embodiment of the present invention.

A breast pump according to the present invention may also be configured such that extracted breast milk is not stored in a storing housing 700 but directly stored in a milk bottle 20. That is, in the breast pump according the present invention, as illustrated in FIGS. 9 to 11, a milk bottle connector 240 which is coupled by screws on the inlet side of the milk bottle 20 may be formed in a cap connector 200 on the side in which a breast milk discharge tube 210 is formed.

As such, when the milk bottle connector 240 is formed so as to surround the breast milk discharge tube 210, the breast milk discharge tube 210 and a silicon valve 600 coupled thereto face the inside of the milk bottle 20 when the milk bottle connector 240 is coupled to the inlet of the milk bottle 20. Thus, the breast milk discharged through the breast milk discharge tube 210 and the silicon valve 600 may be directly filled in the milk bottle 20.

Here, the remaining configuration except for the milk bottle connector 240 is the same as that of the breast pump illustrated in FIGS. 1 to 8, detailed descriptions on the breast milk extraction process and the operation of each of the constituents will not be provided.

So far, although exemplary embodiments of the present invention have been described in detail, the scope of the present invention is not limited to specific embodiments but should be interpreted by attached claims. Furthermore, those skilled in the art should understand that various changes and modifications could be made within the scope of the present invention.

1. A breast pump comprising:
a close-contact housing (100) brought into close contact with a breast (10) of a lactating woman and having
a protruding part (110) which extends forward and is formed on a portion of the close-contact housing (100) which corresponds to a nipple of the lactating woman, and
a breast milk discharge hole (120) formed on an end-side bottom surface of the protruding part (110);
a cap connector (200) coupled in a structure covering an end side of the protruding part (110), having a spacing space (G) between the cap connector (200) and an end-side surface of the protruding part (110), and provided with a breast discharge tube (210) which extends downward from a lower end of the breast discharge hole (120) and communicates with the spacing space (G);
a funnel connector (300) provided on an upper side of the cap connector (200) so as to have a concavely formed upper surface to thereby have an inner space and provided with a communication flow passage (310) through which the inner space and the spacing space (G) communicate with each other;
a cover (400) provided on the funnel connector to cover the inner space of the funnel connector (300);
a flow separation membrane (500) for vertically separating a space between the funnel connector (300) and the cover (400) into a first space between the cover (400) and the flow separation membrane (500) and a second space between the flow separation membrane (500) and the funnel connector (300); and
an air tube (420) through which air is allowed to flow into/out of the first space between the cover (400) and the flow separation membrane (500).

2. The breast pump of claim 1 further comprising a storing housing (700) coupled to the close-contact housing (100) in a detachable structure so as to house therein the cap connector (200), the funnel connector (300), the cover (400), the flow separation membrane (500), and a silicon valve (600), and provided with a space in which breast milk discharged through the breast milk discharge tube 210 is stored.

3. The breast pump of claim 2, wherein
a cleaning hole (220) communicating with the communication flow passage (310) is formed on an outer end of the cap connector (200), and
the storing housing (700) is provided with a sealing packing (710) pressed to the cleaning hole (220) when coupled to the close-contact housing (100).

4. The breast pump of claim 1, wherein the cap connector (200) is provided with a milk bottle connector (240) on a side in which the breast milk discharge tube (210) is formed.

5. The breast pump of claim 1, further comprising a silicon valve (600) having
one side coupled to an outlet side of the breast milk discharge tube (210) and
the other side which is formed to have a sharp edge and provided with a cut line.

6. The breast pump of claim 1, further comprising an electric pump (800) for suctioning air through the air tube (420).

7. The breast pump of claim 2, further comprising a silicon valve (600) having
one side coupled to an outlet side of the breast milk discharge tube (210) and
the other side which is formed to have a sharp edge and provided with a cut line.

8. The breast pump of claim 3, further comprising a silicon valve (600) having
one side coupled to an outlet side of the breast milk discharge tube (210) and
the other side which is formed to have a sharp edge and provided with a cut line.

9. The breast pump of claim 4, further comprising a silicon valve (600) having
one side coupled to an outlet side of the breast milk discharge tube (210) and
the other side which is formed to have a sharp edge and provided with a cut line.

10. The breast pump of claim 2, further comprising an electric pump (800) for suctioning air through the air tube (420).

11. The breast pump of claim 3, further comprising an electric pump (800) for suctioning air through the air tube (420).

12. The breast pump of claim 4, further comprising an electric pump (800) for suctioning air through the air tube (420).
